(19) United States
(12) Patent Application Publication
(10) Pub. No.: US 2006/0183132 A1
(43) Pub. Date: Aug. 17, 2006

(54) Selection probe amplification

(75) Inventors: Glenn Fu, Dublin, CA (US); Laura Stuve, San Jose, CA (US); John Sheehan, Mountain View, CA (US); Amy Ollmann, Redwood City, CA (US); Naiping Shen, Saratoga, CA (US); Andrew B. Sparks, Saratoga, CA (US); Dennis Ballinger, Menlo Park, CA (US)

Correspondence Address: BEYER WEAVER & THOMAS LLP, P.O. BOX 70250, OAKLAND, CA, 94612-0250, US

(73) Assignee: Perlegen Sciences, Inc., Mountain View, CA (US)

(21) Appl. No.: 11/058,432

(22) Filed: Feb. 14, 2005

(51) Int. Cl. C12Q 1/68; C12P 19/34

(52) U.S. Cl. 435/6; 435/91.2

(57) ABSTRACT

Multiple unique selection probes are provided in a single medium. Each selection probe has a sequence that is complementary to a unique target sequence that may be present in a sample under consideration. For example, each selection probe may be complementary to a sequence that includes one of the SNPs used to genotype an organism. Single-stranded selection probes anneal or hybridize with sample sequences having the unique target sequences specified by the selection probe sequences. Sequences from the sample that do not anneal or hybridize with the selection probes are separated from the bound sequences by an appropriate technique. The bound sequences can then be freed to provide a mixture of isolated target sequences, which can be used as needed for the application at hand.

### BACKGROUND

The present invention pertains to methods, probes, apparatus, kits, etc. for selecting, isolating, and/or amplifying pre-specified sequences in a nucleic acid sample. The invention employs multiple selection probes (often thousands) in a single reaction mixture.

Conventionally, Polymerase Chain Reaction (PCR) is used to amplify a pre-specified region or fragment of a nucleic acid sample. Over multiple cycles of denaturing and annealing, PCR generates many additional copies of a fragment. Often, the nucleic acid sample contains many other sequence regions that are excluded from amplification. In such cases, PCR effectively selects or isolates the pre-specified sequence of interest from the remainder of the nucleic acid sequence.

In many applications of interest, PCR is employed to amplify multiple distinct sequences within a nucleic acid sample. This can be an effective tool when the sample contains relatively few sequences to be amplified but it becomes expensive and time consuming when there are many sequences under consideration. Each sequence to be amplified requires its own unique set of PCR primers. These can be expensive to produce or obtain. Further, until recently, each sequence required a separate PCR amplification reaction performed in its own reaction vessel with its own PCR reactants.

Multiplex PCR is a process that addresses some of these difficulties. It amplifies multiple sequences in a single reaction vessel. In multiplex PCR, the vessel includes the sample under analysis, a unique primer set for each sequence to be amplified, as well as polymerase and deoxyribonucleotide triphosphates (dNTPs—e.g., dATP, dCTP, dGTP, and dTTP) to be shared by all amplification reactions. Thus, it has become possible to simultaneously amplify hundreds of sequences in a single reaction mixture. This can greatly improve efficiency. However, it still requires a unique set of primers for each sequence to be amplified and therefore the cost of the procedure is nearly proportional to the number of sequences to be amplified or isolated. Further, there are many applications where far more than a few hundred sequences must be amplified. For example, to fully genotype an individual of a higher species requires amplification of many thousands of sequences. Thus, many separate multiplex PCR reactions must be conducted. Obviously, even with the efficiency gains brought by multiplex PCR, the process can become very costly and time consuming.

The human genome presents a particularly complex sample for analysis. It appears to contain between about five million and about eight million Single Nucleotide Polymorphisms (SNPs). Of these approximately 250,000 are believed necessary to fully genotype an individual. To capture information for this entire set of SNPs requires possibly thousands of different multiplex PCR reactions. This represents a significant practical hurdle to unlocking the therapeutic potential recently achieved by mapping the entire human genome.

More efficient techniques for isolating or selecting multiple sequences from a nucleic acid sample would provide an important advance in the field.

### SUMMARY

The present invention provides an advanced technique for isolating or selecting multiple sequences from a nucleic acid sample by employing multiple unique selection probes in a single medium (typically thousands of such probes). Each selection probe has a sequence that is complementary to a unique target sequence that may be present in the sample under consideration. For example, each selection probe may be complementary to a sequence that includes one or more of the SNPs used to genotype an organism. Methods of this invention allow single-stranded (e.g., denatured, double-stranded) selection probes to anneal or hybridize with sample sequences having the unique target sequences specified by (e.g., complementary to) the selection probe sequences. Sequences from the sample that do not anneal or hybridize with the selection probes are separated from the bound sequences by an appropriate technique. The bound sequences can then be freed to provide a mixture of isolated target sequences, which can be used as needed for the application at hand. For example, the isolated target sequences may be contacted with a nucleic acid array to genotype an organism from which the sample was taken.

One aspect of the invention provides a method of selecting or isolating target nucleic acid sequences from a nucleic acid sample. The method may be characterized by the following sequence of operations: (a) generating nucleic acid fragments from the sample; (b) amplifying the nucleic acid fragments; (c) exposing the amplified nucleic acid fragments to at least about 2000, or at least about 5000, or at least about 10,000 distinct selection probes in a single reaction medium under conditions that promote annealing between the selection probes and the amplified nucleic acid fragments that are complementary to the selection probes; (d) removing the amplified nucleic acid fragments that are not strongly bound to the selection probes; and (e) releasing annealed amplified nucleic acid fragments from the selection probes. In this method, it is understood that the selection probes have sequences complementary or nearly complementary to the target nucleic acid sequences. Thus, the annealed amplified nucleic acid fragments contain the target nucleic acid sequences. The method effectively selects or isolates the target nucleic acid sequences.

The method may contain a further operation of characterizing the nucleic acid sample on the basis of the target nucleic acid sequences released in (e). In one embodiment, this is accomplished by applying the target nucleic acid sequences to a nucleic acid array. To facilitate this, the process may also (i) amplify the target nucleic acid sequences released in (e), and (ii) label the target nucleic acid sequences prior to contacting them with the nucleic acid array. According to another implementation detail, the method further fragments the target nucleic acid fragments prior to labelling and/or contact with the array.

The conditions employed to generate fragments the sample (operation (a)), are chosen to provide fragments of a size and structure appropriate for the remainder of the process. In one embodiment, fragmentation produces nucleic acid fragments having an average length of between about 25 and about 2,000 base pairs or more, and preferably about 500 base pairs. For some processes, the fragmentation produces nucleic acid fragments having an average size that allows genotyping on a microarray without further fragmentation. In some cases, avoidance of a phenomenon known as PCR suppression requires that fragmentation be conducted in two stages, one prior to and the other after amplification (operation (b)).

In a specific embodiment, amplification is accomplished using PCR on substantially all of the nucleic acid fragments produced by the fragmentation operation (a). The process may be designed so that this is accomplished without providing unique primers for each fragment. For example, the process may involve attaching “adaptors” to the ends of the nucleic acid fragments. The adaptors include relatively short sequences complementary to general-purpose primers employed in the PCR amplification. When all adaptors have the same sequence or when the adaptors comprise only a few different sequences, then only one or a few primer sets are needed to amplify all fragments. Stated another way, a limited set of primers can amplify all fragments having the adaptors, without regard to the specific sequences embodied in the fragments. In one specific embodiment, the adaptors are double-stranded sequences with a single-stranded tail or overhang. In another specific embodiment, the adaptors have an additional function: they act as PCR primers in the subsequent amplification operation. In this embodiment, some, but not all, adaptors ligate to sample fragments. Those that remain in solution serve to provide the subsequently needed primers.

In a specific embodiment, amplification is accomplished using PCR on substantially all of the nucleic acid fragments produced from the target nucleic acids prior to further analysis, e.g., through contact with a microarray after operation (e). This embodiment may employ a primer having the same sequence as those used to amplify nucleic acid fragments (in operation (b)), but that instead of excess double-stranded adaptors being used, a single-stranded primer may be added.

The described method separates fragments that bind to selection probes from those that do not. This may be accomplished in many ways. In one approach, the selection probes (which may be single- or double-stranded) bind to a solid substrate, which can be washed or otherwise treated to remove unbound sample fragments. To implement this approach, the selection probes may be initially contacted with the amplified nucleic acid fragments (operation (c)) and then linked to the solid substrate. At least a subset of the selection probes will be annealed to the amplified nucleic acid fragments between operations (c) and (d). To facilitate linking the selection probes to the solid substrate, the probes may include moieties that tightly bind to the solid substrate.

To remove the amplified nucleic acid fragments that are not strongly bound to the selection probes (and are hence not strongly bound to the solid substrate), the process may involve washing the substrate to remove the unbound or weakly bound nucleic acid fragments. In one approach, this involves exposing the solid substrate to a solution under conditions that remove partially annealed amplified nucleic acid fragments from bound selection probes. Such partially annealed amplified nucleic acid fragments may contain one or more mismatches relative to the target sequence and therefore may not be fully complementary to any of the selection probes.

A significant benefit of the invention is the ability to select or isolate thousands of distinct target sequences in a single reaction medium. To this end, the reaction medium may include thousands of sequence specific selection probes; e.g., between about 10⁵ and about 10⁸ such selection probes. Within this range, significant advantages over multiplex PCR can still be realized when using only a few thousand unique selection probes, e.g., at least about 1,000, 2,000, 5,000, 10,000, 50,000, 100,000, 1,000,000 or 10,000,000.

Another aspect of the invention pertains to methods employing a single primer for initial amplification. Such methods may be characterized by the following operations: (a) applying an adaptor sequence to the ends of the target and non-target nucleic acid fragments in the mixture; (b) performing a polymerase chain reaction to amplify the target and non-target fragments, wherein no primer sequence is necessary to amplify the target and non-target fragments besides that provided by denaturation of excess adaptors; (c) contacting the amplified target and non-target fragments with a plurality of selection probes simultaneously, under conditions that promote annealing of the selection probes and the target nucleic acid fragments; and (d) separating the non-annealed and partially-annealed non-target nucleic acid fragments from the annealed target nucleic acid fragments, which are bound to said selection probes, thereby selecting the target nucleic acid fragments. As with the method described above, the selection probes comprise sequences complementary to sequences of the target nucleic acid fragments. Preferably, the adaptor sequence comprises a sequence of between about 15 and 40 base pairs in length and/or is present in excess to the number of fragment ends in the range of about 10- to 100-fold excess.

In one embodiment, the adaptor sequence is a double-stranded nucleic acid sequence. It may have one blunt end and one non-blunt (sticky) end. In this embodiment, the blunt end may be used for attachment to the ends of the nucleic acid fragments. To prevent self-annealing, a double-stranded adaptor having a sticky end may be designed to have an overhang that is not complementary to itself. Further, to prevent self-ligation of adaptors, one strand of the adaptor may lack a moiety necessary for ligation at the blunt end of the adaptor (e.g., a 5′ phosphate group).

Still another aspect of the invention pertains to a set of selection probes for use in simultaneously isolating target nucleic acid fragments from non-target nucleic acid fragments. Such probe set may be characterized as follows: (a) having at least about 1,000, or 5,000 or 10,000 distinct selection probes in a common medium, and (b) wherein each of the distinct selection probes is between about 20 and 1000 base pairs in length. In one embodiment, each selection probe has a sequence complementary to a distinct target sequence including at least one distinct SNP, all found in a single genome. In certain embodiments, each distinct target sequence comprises only one SNP. In other embodiments, each distinct target sequence comprises at least two or more SNPs. In still further embodiments, some target sequences comprise only one SNP, while others comprise two or more SNPs.

The selection probes may be either double- or single-stranded. They may be prepared by various techniques such as specific PCR reactions. The set may include between about 10⁴ and 10⁷ distinct selection probes, or between about 10⁴ and 10⁵ distinct selection probes in a more specific case. In certain embodiments, the selection probes are PCR amplicons between about 50 and 200 base pairs in length.

In a further embodiment, each of the distinct selection probes contains a moiety, apart from the selection probe sequence, that facilitates binding to a solid substrate. As an example, the moiety may be biotin or streptavidin.

Another aspect of the invention provides a kit for selecting target nucleic acid fragments from non-target nucleic acid fragments. Such kit includes (i) a set of selection probes as described above (e.g., at least about 1,000 or 2,000 or 5,000 or 10,000 distinct selection probes in a common medium); and (ii) a solid substrate having a surface feature for binding with the moiety on the selection probes and thereby facilitating immobilization of the selection probes on the solid substrate. As an example, the solid substrate may take the form of beads. Further, the selection probes may include a moiety to facilitate binding to the solid substrate (via the surface feature). In some cases, the kit will also include primers and polymerase for amplifying the nucleic acid fragments. It may also include a microarray comprising sequences complementary to the target nucleic acid fragments.

In a specific embodiment of the invention, the complete sequence of operations involves (1) generating nucleic acid fragments of appropriate size from a genome, (2) adding universal adaptors to both ends of the fragments in order to allow amplification with one primer or a simple primer set, (3) amplifying the fragments, (4) annealing the amplified fragments with selection probes complementary to sequences at SNP locations of interest (the probes contain biotin or other molecular feature that allows affixation to a solid substrate), (5) linking the selection probes (together with the complementary sequences) to a solid substrate, (6) washing the substrate to remove unbound and loosely bound genomic fragments, (7) separating the complementary genomic fragments from the immobilized selection probes by denaturation, (8) amplifying the selected genomic fragments using primers that have the same nucleotide sequence as those that were employed in the initial amplification process, (9) fragmenting the amplified fragments into smaller fragments appropriate for binding with a microarray, and (10) hybridizing the fragments to target probes on the microarray to genotype the genome.

These and other features and advantages of the present invention will be described in more detail below with reference to the associated drawings.

### BRIEF DESCRIPTION OF THE DRAWINGS

FIG. 1 is a process flow chart depicting a specific method for isolating target nucleic acid sequences from a sample in accordance with an embodiment of this invention.

FIGS. 2A and 2B diagrammatically depict fragmentation of a nucleic acid strand into multiple fragments, some of which contain a target sequence of interest.

FIG. 3A depicts the fragments of FIG. 2B with adaptors attached to the ends of the fragments to facilitate subsequent amplification.

FIG. 3B diagrammatically depicts a ligation process for attaching a double-stranded adaptor to a blunt end of a nucleic acid fragment.

FIG. 3C shows an adaptor structure in which blunt ends of the adaptors are designed to lack a linking moiety (e.g., a phosphate group) and thereby prevent self-ligation.

FIG. 3D diagrammatically depicts polymerization of a fragment strand with attached adaptors to remove adaptor sequences beyond nick positions in a double-stranded structure.

FIG. 4A depicts a medium in which selection of target sequences can be accomplished through use of selection probes.

FIG. 4B depicts the medium of FIG. 4A after treatment to denature the initial sequences and then reanneal them under conditions promoting binding between single-stranded selection probes and single-stranded target nucleic acid fragments.

FIG. 5 diagrammatically depicts immobilization to a solid substrate of double-stranded nucleic acids containing selection probes.

FIG. 6 shows three examples of the alignment between a selection probe and a SNP position in a target nucleic acid sequence.

FIG. 7 depicts two different scenarios by which a sample nucleic acid fragment may be “bound” to a selection probe, in one case tightly bound and in another case loosely bound.

FIG. 8 depicts the process of amplifying and further fragmenting the isolated target nucleic acid sequences.

FIG. 9 diagrammatically depicts contacting the isolated target sequences with a nucleic acid array such as a DNA microarray.

### DESCRIPTION OF A PREFERRED EMBODIMENT

Introduction and Overview

The present invention employs a single medium containing at least about 1000, 2000, 5000, 10,000, 30,000, 50,000, 80,000, 100,000, 1,000,000, or 10,000,000 distinct selection probes. Each selection probe has a sequence complementary to a distinct target of interest, such as the sequence associated with a particular SNP. Using the selection medium, fragments of a nucleic acid sample (e.g., genomic DNA) are allowed to anneal with selection probes and thereby become “selected.” Thus, in a single step using a single medium, thousands of target fragments are concurrently selected from the non-target fragments in the sample. This method compares favorably with multiplex PCR, where only a few hundred selective amplifications can occur simultaneously in a single reaction medium. In short, the invention efficiently enriches target sequences in very complex nucleic acid samples.

The selection medium itself represents an advance in the art. In one example, it contains at least about 10,000 different selection probes, each about 50 to 500 base pairs in length and containing a moiety that facilitates linkage to a solid substrate, thereby facilitating separation of annealed target fragments from un-annealed non-target fragments.

Another point of interest, which will be explained in more detail below, is use of a universal adaptor sequence, which allows a single primer to amplify all of the many thousands of nucleic acid fragments generated from a genomic sample. The simultaneously amplified sample fragments will have many different sequences. If a second amplification is employed later in the process, the same single primer can be used again. For example, if target fragments selected by binding to the selection probes are to be further amplified, the same primer may be used to separately amplify those target fragments.

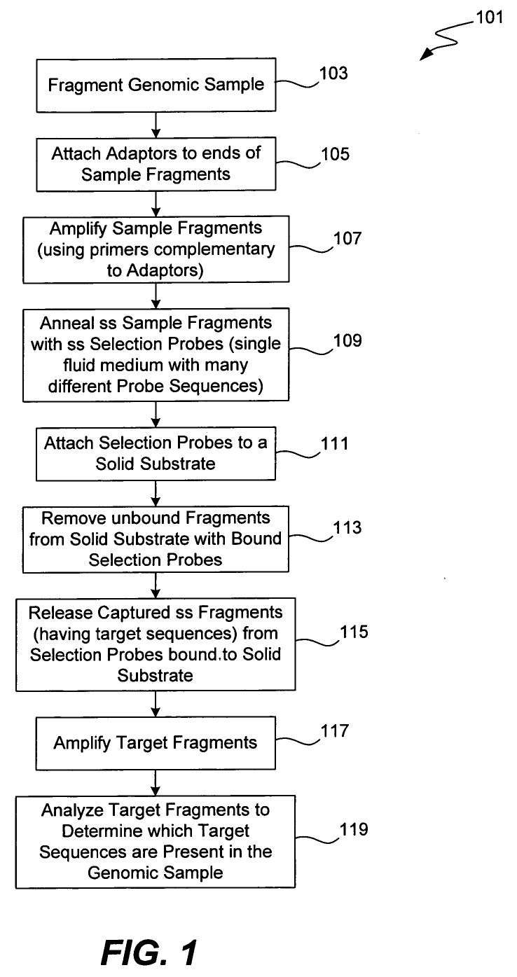

A general outline of a sequence of operations for an exemplary method of this invention is depicted in FIG. 1. As shown there a reference number 101 identifies the overall method, which begins with fragmentation of a nucleic acid sample (e.g., a complex genomic sample). See operation 103. As explained below, various fragmentation techniques may be employed for this purpose. The one chosen for a given implementation will produce fragments of a desired size range and end structure.

Next, as depicted in a block 105, the adaptors are attached to the sample fragments generated in operation 103. Adaptors are employed to permit amplification of all fragments, regardless of sequence, using a limited number of primers, in some embodiments only one. The adaptor has a sequence chosen to be complementary to the primer. As explained below, excess adaptors in solution can, in some embodiments, serve as the primers themselves. After the adaptors have been attached, the sample is amplified as indicated at a block 107. Typically, this involves a PCR process with the appropriate primers, e.g., free adaptor sequences.

Next, in an operation 109, the amplified sample fragments are denatured to produce single-stranded sequences which are subsequently annealed with a large collection of selection probes, each having a sequence complementary to a specific target sequence to be isolated from the genomic sample. Selection probes may be introduced in single-stranded form, or may be introduces in double-stranded form and denatured simultaneously with the amplified sample fragments. As indicated above, a single fluid medium contains many different probe sequences, often many thousands of different probe sequences. This allows much more efficient selection of target sequences than was afforded by prior techniques.

After the annealing process concludes, many of the single-stranded selection probes will have annealed with complementary target fragments from the sample to produce double-stranded nucleic acid sequences. These are then attached to a solid substrate as indicated at block 111. In one embodiment, the selection probes contain a moiety that facilitates linking to a solid substrate, thereby limiting immobilization to nucleic acids containing at least one single strand from the selection probes.

Next, as indicated at a block 113, unbound fragments are removed from the solid substrate. Of course, the substrate will still contain immobilized selection probes, some of which are annealed with complementary genomic fragments. Removal operation 113 may employ a defined washing protocol such as the one described below.

The next operation in process 101 involves releasing captured single-stranded fragments (which have target sequences) from selection probes linked to the solid substrate. This may simply involve exposing the solid substrate to conditions that denature the bound double-stranded fragments. Because only the selection probes contain moieties linking them to the solid substrate, the captured target fragments are free to reenter solution for further analysis. Before such analysis, the target fragments may be optionally amplified as indicated at block 117. And, depending on the analysis technique, the fragments may need to be further fragmented to a smaller size to facilitate their capture, handling and further analysis. Finally, as indicated at a block 119, the isolated target fragments are further analyzed, e.g., to determine exactly which target sequences are present in the genomic sample. As indicated, this may be accomplished using a microarray of immobilized nucleic acid sequences. Other techniques such as direct sequencing may be employed as well.

Not all of the operations in process 101 are necessary in all implementations of the invention. For example, some embodiments may hybridize sample fragments with pre-immobilized single-stranded selection probes. In such embodiments, the selection probes are provided with the solid substrate (e.g., beads, columns, microarrays, etc.) to which they are immobilized. In this case, the target sample fragments will hybridize with single-stranded selection probes already on the solid substrate. No separate step of attaching the probes hybridized to the target fragments to the solid substrate is required in this embodiment. Obviously, the probes may be attached to the substrate in a separate operation, prior to hybridization. Other specific steps from the process can be generalized. Thus, an alternative characterization of the method involves the following: (1) fragmenting a nucleic acid sample to produce multiple nucleic acid fragments; (2) annealing or hybridizing the amplified nucleic acid fragments with selection probes having sequences complementary to genomic sequences proximate to SNPs or other features of interest; (3) separating nucleic acid fragments that are not bound to the selection probes from those that are; and (4) genotyping the target nucleic acid fragments that were previously bound to the selection probes, thereby selectively genotyping the nucleic acid sample only at the loci of interest (e.g. SNPs).

The Sample and its Fragments

As indicated, processes of this invention act on nucleic acid samples. The samples will have target and non-target sequences. The process enriches the sample by selecting or isolating the target sequences. In so doing the process may also amplify the target sequences. Generally, the invention provides its greatest advantages over current technologies in situations where there are at least a few hundred or a few thousand or tens of thousands of distinct target features or sequences found within a complex sample.

The nucleic acid sample is obtained from an organism under consideration and may be derived using, for example, a biopsy, a post-mortem tissue sample, and extraction from any of a number of products of the organism. In many applications of interest, the sample will comprise genomic material. The genome of interest may be that of any organism, with higher organisms such as primates often being of most interest. Genomic DNA can be obtained from virtually any tissue source. Convenient tissue samples include whole blood and blood products (except pure red blood cells), semen, saliva, tears, urine, fecal material, sweat, buccal, skin and hair. The nucleic acid sample may be DNA, RNA, or a chemical derivative thereof and it may be provided in the single or double-stranded form. RNA samples are also often subject to amplification. In this case amplification is typically preceded by reverse transcription. Amplification of all expressed mRNA can be performed, for example, as described by commonly owned WO 96/14839 and WO 97/01603.

In a specific embodiment, the target features of interest are relatively short sequences containing SNPs. As indicated above, in the case of the human genome, there are between about five million and about eight million known SNPs. This invention provides a method for efficiently isolating and amplifying sequences associated with such SNPs. Other target features (aside from SNPs) that can be isolated using the invention include insertions, deletions, inversions, translocations, other mutations, microsatellites, repeat sequences—essentially any feature that can be distinguished by its nucleic acid sequence. These features may occur, e.g., in exons or other genic regions, in promoters or other regulatory sequences, or in structural regions (e.g., centrosomes or telomeres). Regardless of whether SNPs or other features serve as targets, the invention finds use in a broad range of applications including pharmaceutical studies directed at specific gene targets (e.g., those involved in drug response or drug development), phenotype studies, association studies, studies that focus on a single chromosome or a subset of the chromosomes comprising a genome, studies that focus on expression patterns employing, e.g., probes derived from mRNA, studies that focus on coding regions or regulatory regions of the genome, and studies that focus on only genes or other loci involved in a particular biochemical or metabolic pathway. In other words, target sequences may be selected and isolated from a sample based on many different criteria or properties of interest. In other examples, target sequences are selected based on how the target sequences will be further analyzed and processed, e.g., based on the design of a DNA microarray to which the target sequences will be applied.

As explained, the original nucleic acid sample may be fragmented to produce many different nucleic acid fragments, some of them harboring a target feature or sequence of interest and others not. Of course, it is possible that the initial sample will be provided in fragmented form of appropriate size and condition, which requires no separate fragmentation operation. All fragments (target fragments and non-target fragments alike) will typically possess certain common features such as general size ranges and end characteristics (e.g., blunt versus sticky). The population of fragments may be further characterized by an average size and a size distribution, as well as an occurrence rate of the target sequence. The fragmentation conditions determine these characteristics.

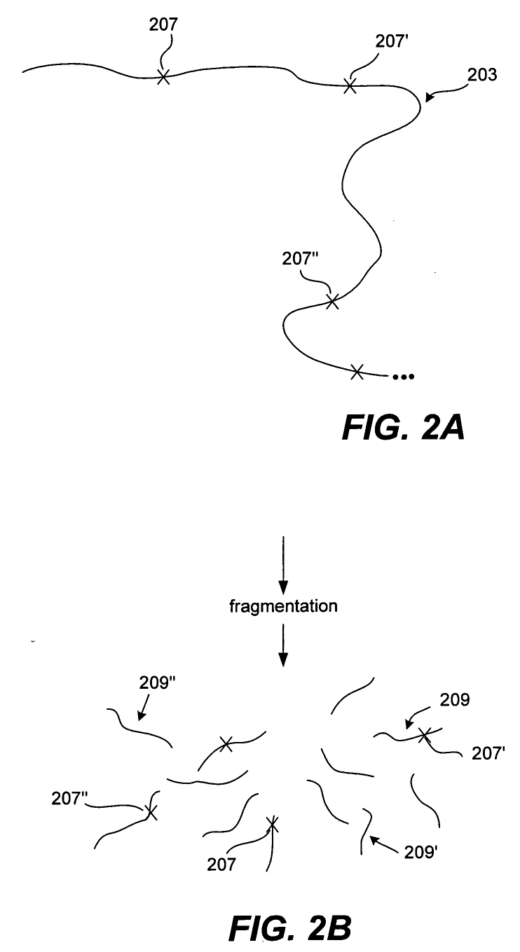

FIG. 2A depicts a continuous strand of nucleic acid 203 that may form part of a sample to be analyzed; e.g., a double-stranded segment of genomic DNA taken from a human donor. Strand 203 is shown to have multiple target features 207, 207′, 207″, . . . . These may represent SNPs or other features under investigation. At operation 103 in method 101, the sample is fragmented. This is depicted in FIG. 2B, where continuous strand 203 is fragmented into multiple strands 209, 209′, 209″, etc. Some of these strands, such as strand 209, contain a target feature of interest. Other strands such as strands 209′ and 209″ contain no target sequence. As explained, when nucleic acid fragments are processed in accordance with this invention many or most of the target containing fragments are separated from many or most of the non-target containing fragments.

Various considerations come into play when selecting an average or mean fragment length. In a typical case, the mean fragment size is between about 20 and 2000 base pairs in length or even longer, but preferably between about 50 and 800 base pairs in length. In certain embodiments, the mean fragment size is between about 400 and 600 base pairs in length. In other embodiments, the mean fragment size is between about 100 and 200 base pairs in length. As one of skill will readily recognize, the optimal mean fragment length may depend on the specific application. For example, the fragment must be large enough to contain unique sequence. If hybridization will be used to select or analyze the target sequences, the fragment must be large enough to hybridize well with its complementary sequence in the particular hybridization conditions. The fragments should be small enough so that they are not easily sheared during subsequent manipulations, and so that they do not interfere with hybridization to the selection probes. Further, they should be of an appropriate size as required by the subsequent manipulations, e.g., long-range PCR, short-range PCR, etc.

Another factor to consider in determining an appropriate fragment length is the final sequence analysis technique to be considered. For example, if a nucleic acid microarray is employed, the desired fragment size will be approximately 25 to 100 base pairs. If the initially produced fragments are significantly larger than this, a second fragmentation must be performed prior to genotyping with a microarray. Ideally, the initial fragmentation would produce fragments of a size suitable for analysis so that no further fragmentation would be necessary. Unfortunately, it has been found that fragments of 25 to 100 base pairs in size may exhibit “PCR suppression.” This results when the primer-complementary ends of a given fragment bind to one another in a single strand to form a hairpin structure. Such hairpin structures cannot participate in the PCR amplification. Only when the fragments are significantly larger (e.g., greater than at least about 300 base pairs) is the probability of the end to end binding of a single strand reduced to a point where PCR suppression is not a significant concern.

One might minimize the likelihood that these hairpin structures will form by employing two different adaptor sequences which are not complementary to one another. For example, the use of adaptor sequences A and B will result in approximately one quarter of the ligated products having two A adaptors, approximately one quarter of the ligated products having two B adaptors, and approximately one half of the ligated products having one A and one B adaptor. Thus, a significant fraction of the resulting ligated products will still be susceptible to PCR suppression.

To facilitate attachment of adaptor sequences, the fragment ends preferably have a consistent structure, e.g., either all blunt or all sticky. In the later case, all sticky ends preferably have the same overhang sequence in order to provide a consistent structure for attachment to corresponding adaptor ends. In a preferred embodiment, however, the fragments are blunt-ended. A specific embodiment in this invention, which is detailed below, employs fully blunt-ended adaptors.

Fragmentation of the sample nucleic acid can be accomplished through any of various known techniques. Examples include mechanical cleavage, chemical degradation, enzymatic fragmentation, and self-degradation. Self-degradation occurs at relatively high temperatures due to DNA's acidity. The fragmentation technique can provide either double-stranded or single-stranded DNA. U.S. patent application Ser. No. 10/638,113, filed Aug. 8, 2003, describes various methods, apparatus, and parameters that can be controlled to provide desired levels of fragmentation. That application is incorporated herein by reference for all purposes.

Enzymatic fragmentation is accomplished using a nuclease such as a DNAse. In one example, DNaseI is used in the presence of manganese (II) ions. Cleavage with this enzyme gives relatively blunt-ended double-strand fragments. Still there may be a one or two base overhang in the resulting fragments. In such cases, fully blunt-ended fragments can be produced from the moderately sticky ended fragments by treatment with certain exonucleases such as that exhibited by Pfu DNA polymerase. The Pfu enzyme acts by trimming back 3′ extensions on both ends of the DNA fragments. It also fills in 3′ recessive ends by polymerase activity. Other methods for generating blunt-ended fragments include mechanical shearing and acid hydrolysis both of which produce some blunt ends and some overhangs. Thus the fragments will still require some “blunting” as with Pfu polymerase. Further, certain restriction enzymes that leave blunt ends (e.g., AluI, HaeIII, HinDII, SmaI) can be employed. Other restriction enzymes that leave overhangs which can be “blunted” may also be used. Of course, any of the techniques which leave sticky ends (including random overhang sequences) can be used without subsequent blunting so long as the process uses compatible adaptors (e.g., ones with random ends so that no matter what the overhang was it would still get an adaptor).

Adaptors and Amplification

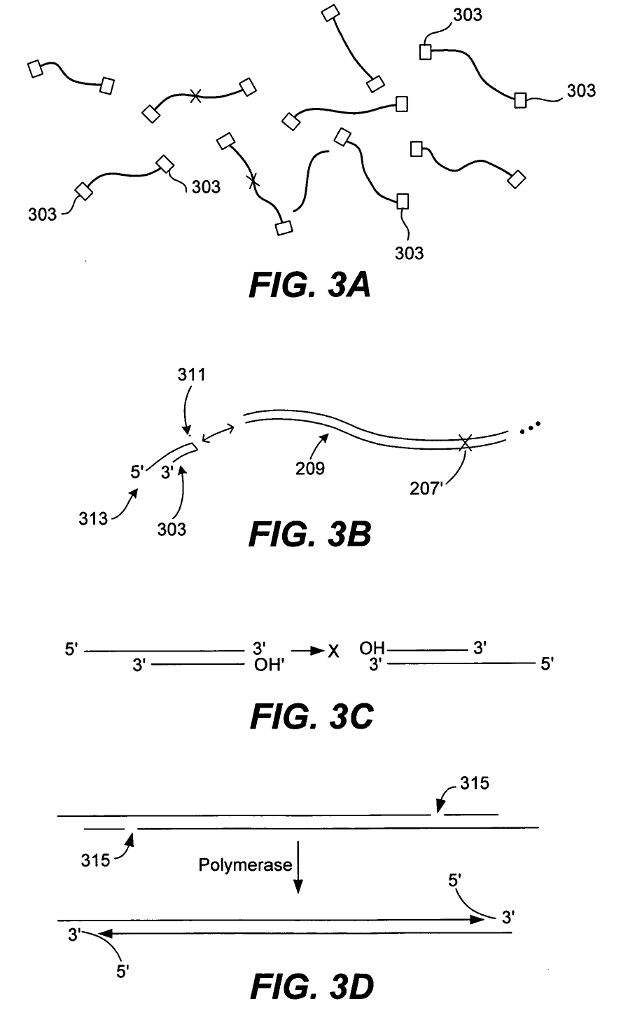

To amplify the sample fragments but avoid the cost of preparing or purchasing many different primers, the invention optionally employs one or more universal adaptor sequences. These adaptors are attached to both ends of all sample fragments where they provide common sequences for primer annealing. See block 105 of FIG. 1. See also FIG. 3A, which depicts in cartoon fashion the fragments of FIG. 2B after adaptors 303 have been attached. Preferably only a single adaptor sequence is provided for attachment to all the many fragments produced from a sample. With this approach only one primer sequence is needed to amplify all fragments. In alternative embodiments, more than one adaptor sequence is employed, but generally it will be advantageous to employ no more than a few. This section describes both the structure of the adaptors and a method of attaching them to the fragments.

The adaptors should have a length that is appropriate for their purpose: i.e., to provide a site for annealing with a PCR primer. Thus, the adaptors are typically about 25 to 50 base pairs long. In one preferred embodiment, they are double-stranded with one blunt end and one sticky end. As explained below, this allows the adaptor to bind to the fragments in a consistent orientation and it also permits excess adaptors to serve as PCR primers during subsequent amplification. Of course, the invention is not limited to this structure, and in some cases the adaptors may be single-stranded sequences.

In many cases, the concentration of the adaptor should be well in excess of the fragment concentration. This ensures that there will be sufficient adaptors available to promote rapid fragment-adaptor ligation. It also reduces the likelihood of fragment-to-fragment ligation. In one embodiment, the adaptor concentration is between about 10- to 100-fold excess over the concentration of fragment ends (which is normally double the concentration of fragments). At this concentration, the unreacted excess adaptor sequences can server as primers for the subsequent amplification. During denaturation, the double-stranded adaptors will separate into single-stranded sequences, one of which can then serve as a primer when annealed to its complementary sequence on the single-stranded fragments.

In the embodiment depicted in the FIG. 3B, the adaptor 303 includes a sticky end 313 and a blunt end 311. The blunt end always attaches to the DNA fragment 209 and the sticky end always faces away from the fragment. Because, the sticky end 313 will not ligate with the blunt-ended fragments, the adaptor is forced to attach in a single orientation dictated by the blunt end to blunt end ligation between the fragment and adaptor. In the example shown, sticky end 313 has a 3′ recess. Ligation may be accomplished with a conventional DNA ligase.

Precautions may be taken to reduce or eliminate self-ligation between adaptors. A blunt end of one adaptor will not link to the sticky end of another adaptor, but it is possible that the blunt ends of two adaptors will link. It could also be possible for sticky ends of two adaptors to link, but only if the overhangs of the adaptors are complementary to one another. This possibility can be eliminated by designing adaptors with non-complementary overhangs. To prevent self-ligation of the adaptors at their blunt ends, the blunt ends may be designed so that one of the single strands contains a chemical feature that renders it unable to link with an adjacent strand in the blunt end of an aligned adaptor.

For example, the 5′ strand in the blunt end of the adaptor may lack a phosphate group. If the blunt ends of two such adaptors were aligned in a manner to promote ligation, the appropriate DNA ligase would be unable to ligate them as each strand would be lacking a phosphate bridge between the two adaptors. Note that the 5′ end of a DNA strand typically has a free phosphate group for ligating with a 3′ hydroxide group. Such binding creates a continuous strand. If the 5′ phosphate group is lacking from one of the blunt end terminal strands of the adaptor, it cannot form a continuous strand. In such cases, it will be impossible to ligate two adaptors as each 5′ to 3′ coupling of the single strands will be prevented. This situation is depicted in FIG. 3C where adaptors 303a and 303b each have a blunt end at which the 5′ strand lacks a phosphate group. When these adaptors are aligned end-to-end as shown, it is impossible for them to ligate because no continuous single strand can form, either between the top strands or the bottom strands. It should be understood that the missing phosphate moiety is but one approach to preventing self-ligation and various chemical blocking mechanisms may be employed. For example, a similar embodiment employs adaptors in which the 3′ OH is missing in the blunt end, instead of the 5′ phosphate.

When the blunt end of an adaptor lines up with the blunt end of a DNA fragment, only one of the single strands is prevented from ligating. The strand with a 5′ end donated by the DNA fragment will have a phosphate group, which allows ligation with the 3′ end of one of the single strands on the adaptor sequence. The resulting ligated product will, however, have a nick 315 at the interface with each adaptor. See FIG. 3D. The adaptor sequence beyond the nick can be replaced with a fully continuous single strand propagating outward from the genomic fragment by a polymerase reaction as shown in the lower portion of FIG. 3D.

In one embodiment, the Pfu DNA polymerase remains present in the reaction mixture during ligation of the adaptors. Because the Pfu DNA polymerase is a thermophilic enzyme, it may be activated by raising the temperature of the mixture (to e.g. about 68° C.). In the presence of dNTPs, the Pfu polymerase will fill in 3′ recesses and possesses strand displacement activity. As such, it acts on the fragments containing the adaptors by initiating DNA polymerization at the nick left due to the lack of a 5′ phosphate, thereby extending the 3′ end of the fragment and displacing the strand of the adaptor lacking the 5′ phosphate as depicted in FIG. 3D. This results in the production of a nick-free double-stranded sequence comprising two adaptor sequences straddling the DNA fragment. Self-ligation between blunt ends of genomic fragments is generally avoided because the concentration of adaptors is so great in comparison to the concentration of nucleic acid fragments that the probability of fragment-to-fragment ligation is minimal.

After the nucleic acid fragments have been modified with adaptors, they can be amplified as indicated above. See block 107 of FIG. 1. A primer or set of primers that is complementary to the adaptor or adaptors is provided to the solution containing the fragments. As indicated, excess adaptor sequences may themselves serve as the primers, in which case no additional primers need be added. Other components necessary for amplification may be provided as necessary (e.g., particular polymerases, dNTPs, buffers, etc.). In the specific embodiment described above, the Pfu polymerase remains in solution and participates in the PCR alone or together with another polymerase such as “Klentaq1” available from AB Peptides, Inc. of St. Louis, Mo., or other polymerases known in the art. PCR amplification is then performed to amplify all of the fragments. In a specific embodiment, the amplification is performed for about twenty cycles, but this is by no means a minimum or maximum requirement. The resulting DNA sequences will have the adaptor sequences straddling the individual DNA fragments produced in operation 103. In some embodiments, the fragment concentration after amplification is between about 1 μg to 1 mg total yield.

The PCR method of amplification is described in PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202, each of which is incorporated by reference for all purposes. The amplification product can be RNA, DNA, or a derivative thereof, depending on the enzyme and substrates used in the amplification reaction. Certain methods of PCR amplification that may be used with the methods of the present invention are further described, e.g., in U.S. patent application Ser. No. 10,042,406, filed Jan. 9, 2002; U.S. Pat. No. 6,740,510 issued on May 25, 2004; and U.S. patent application Ser. No. 10/341,832, filed Jan. 14, 2003, each of which is incorporated herein by reference for all purposes.

Other methods exist for producing amplified sample fragments that may be employed with this invention (e.g., for isolation with selection probes). Some of these techniques involve other methods of tagging nucleic acid fragments, e.g., DOP-PCR, tagged PCR, etc., and are discussed in great detail in Kamberov et al. US2004/0209298 A1, which is incorporated herein by reference for all purposes.

Selection and Isolation of Target Fragments

After amplification of the sample fragments, multiple oligonucleotide selection probes are added to the mixture. Preferably, at least about 1000 or 2000 or 5000 or 10,000 or 30,000 or 50,000 or 80,000 or 100,000, 1,000,000, or 10,000,000 distinct sequences are provided as selection probes in the mixture (approximately 85,000 probes were employed in one example). As explained, the selection probes are brought into contact with the amplified nucleic acid fragments in a single reaction medium and exposed to conditions promoting annealing between the selection probes and the amplified nucleic acid fragments that are complementary to the selection probes.

Each sample probe has a sequence complementary to a target sequence that is believed to be present in the sample (or at least believed to be potentially present). Thus, if 1000 probes are used, 1000 target sequences may be selected. As such, only sample fragments possessing the target sequences will bind with a selection probe and ultimately be isolated from the sample mixture. The probe sequence may be of any length appropriate for uniquely selecting a target sequence. In the case of target SNPs, appropriate lengths range from about 20 to 1000 base pairs, more preferably between about 20 and 200 base pairs (e.g., about 80 base pairs). Other size ranges may be appropriate for other applications.

The selection probes may be single-stranded or double-stranded and may comprise RNA, DNA, or a derivative thereof. In some embodiments discussed below, single strands of the selection probes include a chemical moiety or other feature that facilitates binding to a solid substrate. Functionally, a “probe” is a nucleic acid capable of binding to a target nucleic acid of complementary sequence through one or more types of chemical bonds, usually through complementary base pairing, usually through hydrogen bond formation. A nucleic acid probe may include natural (i.e. A, G, C, or T) or modified bases (e.g., 7-deazaguanosine, inosine). In addition, the bases in a nucleic acid probe may be joined by a linkage other than a phosphodiester bond, so long as it does not interfere with hybridization. Thus, nucleic acid probes may be peptide nucleic acids in which the constituent bases are joined by peptide bonds rather than phosphodiester linkages.

Typically, the annealing mixture will contain multiple copies of each selection probe. Preferably, the concentration of each selection probe in the mixture will be between about 1-100 ng in a 100 μl reaction mixture, and the concentration of fragments will be between about 1-10 μg in a 100 μl reaction mixture.

Broadly the invention may employ any number of distinct selection probes. It is expected that many applications of interest will employ at least about 1000 distinct selection probes, e.g., between about 10⁴ and 10⁷. A more specific quantity contemplated for use in this invention is at least about 2000 distinct probes, and an even more specific amount is at least about 5000 or at least about 10,000 or at least about 50,000 distinct probes. All the selection probes are used in a single solution or mixture which is contacted with all the sample fragments so that selection of thousands of distinct target sequences can take place simultaneously, in a single reaction mixture. For complex samples employing tens or hundreds of thousands of distinct target sequences, about 10,000 to 100,000 or even to 1,000,000 distinct probes may be employed. Preferably, though not necessarily, all selection probes are provided in a single solution or mixture.

Thus, one embodiment of the invention provides a set of selection probes for use in simultaneously selecting target nucleic acid fragments from non-target nucleic acid fragments. The set includes at least about 1000 (preferably at least about 10,000) distinct selection probes in a common medium. As indicated, each selection probe has a sequence complementary to a distinct target sequence such as a sequence associated with a distinct SNP. Preferably any given selection probe will be complementary to a sequence having only a single SNP. All target sequences may be found in a single sample such as a genome. The medium used to contain the probe set will be a buffered aqueous solution. In a specific embodiment, the solution contains approximately 1M Na++ salt, preferably with 50% formamide and 10% dextran sulfate.

Because the set of selection probes represent targets within a larger genome that contains both target and non-target sequences, the selection probes of the common medium contain few if any non-target sequences, or at least they contain only an amount that does not significantly impair the ability of the probes to select their target sequences. At a minimum, the common medium will contain a significantly enriched amount of selection probes complementary target sequences in comparison to non-target sequences (when compared to the relative amounts of target and non-target sequences in the native genome or other sample). This is true whether the relative amount of target-specific selection probes to non-target sequences is measured on the basis of the number of different target-specific probe sequences to number of different non-target fragment sequences or the total number of target-specific probe sequences to the total number of non-target fragments in solution.

Further, a set of selection probes need not contain probes for each and every target sequence identified as relevant to the characterization of the sample. For example, 50,000 distinct SNP alleles may be identified as relevant to the characterization of a sample, but the selection probe set may contain probes to only 40,000 of these alleles. It is within the scope of this invention to apply 40,000 member probe set to the sample mixture in order isolate at least a fraction of the target sequences potentially present in the sample. Further, a probe set may contain more target sequences than are present in a particular sample. For example, a sample may be derived from mRNA from a particular tissue so any target sequence that is not expressed in that tissue will not be present in the sample.

The selection probes may be produced by any appropriate method including oligonucleotide synthesis techniques and isolation from organisms. In the latter case, PCR or other amplification technique may be employed to produce the probe in relatively high concentrations. In a specific example, probes are obtained using PCR (or multiplex PCR) on sequences of the human genome found to hold specific SNPs. In such situations, the individual selection probes may be prepared by PCR reactions using primers specific for such probes. Such genomic sequences may be identified by any method known in the art, e.g., through association studies, linkage analysis, etc.

Many service providers make custom probes available on a contract basis. Selection probes for use with this invention may be ordered from such providers, some of which are the following: Agilent Technologies of Palo Alto, Calif., NimbleGen Systems, Inc. of Madison, Wis., SeqWright DNA Technology Services of Houston, Tex., and Invitrogen Corporation of Carlsbad, Calif. In another approach, the selection probes may be produced by fragmenting genomic DNA (e.g., a single chromosome or clone(s) from a genomic library) known to have target features. Still further, the selection probes may be created from mRNA by conversion to cDNA to select expressed target sequences. In other words, the expressed mRNA possesses the target sequences.

As indicated the selection probe may also include a moiety that facilitates linking to a solid substrate after the annealing process is complete. Examples of such moieties include modification of the DNA to include biotin, avidin, fluorescent dyes, digoxigenin, or other nucleotide modifications. In a specific example, the moiety is biotin or streptavidin, with the substrate surface having streptavidin or biotin, respectively. In alternative embodiments, the selection probes will be provided pre-linked to the solid substrate. In such embodiments, the solid substrate is contacted with the solution of amplified fragments and under conditions promoting hybridization. No separate linking step is required.

Aspects of the invention pertain to kits containing a set of selection probes as identified above together with one or more other items that facilitate enrichment and/or analysis of the target sequences. In one embodiment, the kit also includes a solid substrate (e.g., beads, microarray, column, etc.) having a surface feature for binding with the moiety on the selection probes and thereby facilitating immobilization of the selection probes on the substrate. The kit may also include primers and polymerase for amplifying the nucleic acid fragments. Still further, the kit may be provided with a nucleic acid array or other tool for identifying target sequences contained within the target fragments.

In accordance with embodiments of this invention, the complete set of selection probes and the sample fragments are provided in a single reaction mixture. To promote formation of hybrid annealing products, the relative concentrations of these two components are preferably about 100-fold to about 10,000-fold more fragments than selection probes and more preferably about 500-fold to about 5000-fold more fragments; e.g., about 1000-fold more fragments than selection probes. Note that many applications will employ subsets of a larger “complete” set of selection probes. For example, an association study may link certain SNPs to a condition of interest. A “complete” probe set may include hundreds of thousands or even millions of distinct selection probes for SNP alleles, while the probe set employed for the condition of interest employs only a few thousand of these selection probes.

To actually select the target fragments, the process must provide both the fragments and the selection probes as single strands. So if either of these are present in a double-stranded form, the process begins by first denaturing the double-stranded sequences in the mixture. The conditions in the mixture are then gradually changed to drive annealing. In some implementations, the temperature is changed in a step-wise fashion to promote annealing. In a typical implementation, the annealing takes place for about 10 to 50 hours (36 hours in a specific implementation).

In one embodiment, double-stranded probes and double-stranded fragments are denatured using a 50% formamide solution at a temperature of about 94° C. for about two minutes. Note that an increase of 1% in formamide concentration lowers the melting temperature of double-stranded DNA by about 0.6° C., so the combination of temperature and formamide concentration can be tailored as needed. After denaturing, the sequences are annealed by a slow cool process with certain gradation as described here. Initially, the mixture is cooled from 94° C. to about 42° C. over a period of about 2 hours. Then, the temperature is held at 42° C. for about 12 hours. Thereafter, the solution is slow cooled from 42° C. to about 37° C. over a period of about 5 hours. It is in this temperature range (about 37 to 42° C.) that most of the annealing takes place. After reaching 37° C., the mixture is held at this temperature for about 12 hours. Of course, the invention is not limited to these denaturing conditions. For example, it may be possible to anneal over significantly shorter periods of time, possibly as short as 12 hours.

Generally, annealing refers to the binding, duplexing, or hybridizing of a molecule only to a particular nucleotide sequence under stringent conditions when that sequence is present. Stringent conditions are conditions under which a probe hybridizes to its target subsequence, but to no other sequences. Stringent conditions are sequence-dependent and vary by circumstance. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence anneal to the target sequence at equilibrium. (As the target sequences may be present in excess, at Tm, 50% of the probes are theoretically occupied at equilibrium.) Typically, stringent conditions include a salt concentration of at least about 0.01 to 1.0 M Na ion concentration (or other salts) at pH 7.0 to 8.3 and the temperature is at least about 30° C. for short probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations.

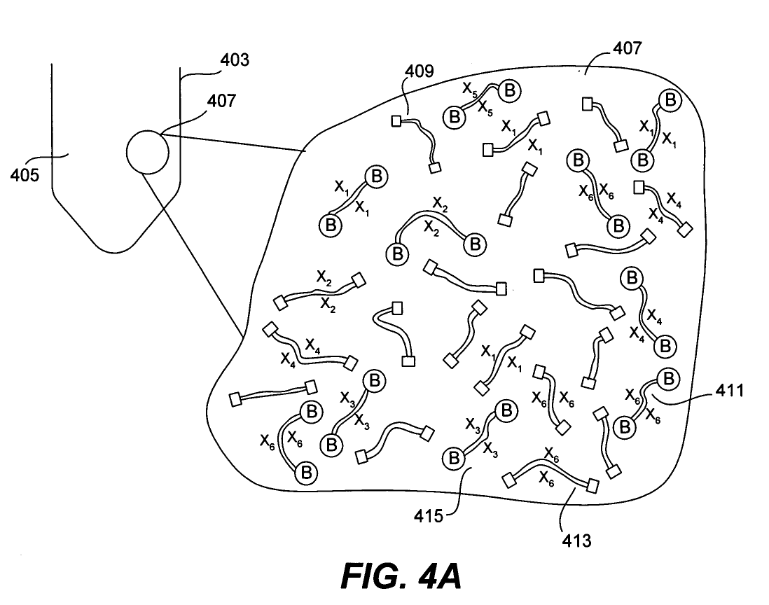

The starting and ending points of the selection process are depicted schematically in FIGS. 4A and 4B. As shown, each of these represents a molecular scale volume 407 of the reaction mixture 405 provided in a single vessel 403. Volume 407 from FIG. 4A has numerous double-stranded species. Selection probes are identifiable by the attached “B” species for biotin. These include probes 411 and 415. In addition, each selection probe will include a target sequence indicated by an “X.” The sample fragments are identifiable by the rectangular adaptor sequences at the ends. Some of the fragments have target sequences X (e.g., fragments 413) while other fragments do not (e.g., fragments 409).

In the idealized example of FIG. 4A, the selection probes hold target sequences X1 through X6. The sample fragments hold only target sequences X1, X2, X4, and X6. Sequence X3 and X5 are not present in the sample. After annealing, as depicted in volume 407′ of FIG. 4B, some probes have hybridized with target fragments and others have not. As shown, sample fragments such as fragment 409, which does not have a target sequence, remains intact. The same is true of the selection probes having targets X3 and X5, as well as probe 411 which holds target X6. This probe did not anneal with the sample fragment 413, which also holds target sequence X6. Of course, some fraction of the complementary selection probes and target fragments will not anneal with each other. In the depicted example, fragments with targets X1, X2, and X4 cross-annealed. Of course, normally there will be multiple copies of the fragments holding the targets, as well as multiple copies of the complementary selection probes. Thus, while typically not all complementary strands will find and anneal to one another, under the proper conditions a significant fraction will anneal to produce probe-sample double-stranded products.

After the sample fragments and selection probes have annealed, they are immobilized by exposing the solution to a solid substrate having an affinity for the selection probes. As indicated, the selection probes can include a moiety that links with a complementary moiety on the substrate surface (e.g., biotin and streptavidin). The solid substrate may take many different forms including beads, disks, columns, microarrays, porous glass surface, membranes, plastics. In a specific embodiment, the substrate comprises beads of approximately 1 micron diameter, each having approximately 10⁵-10⁷ probes per 1 micron bead. Magnetic beads coated with streptavidin, available from Dynal (Oslo, Norway), are suitable for immobilizing biotin-labeled DNA. Procedures for performing enrichments of nucleic acids using immobilized DNA on beads are described by Birren et al., at ch. 3, which is incorporated herein by reference for all purposes.

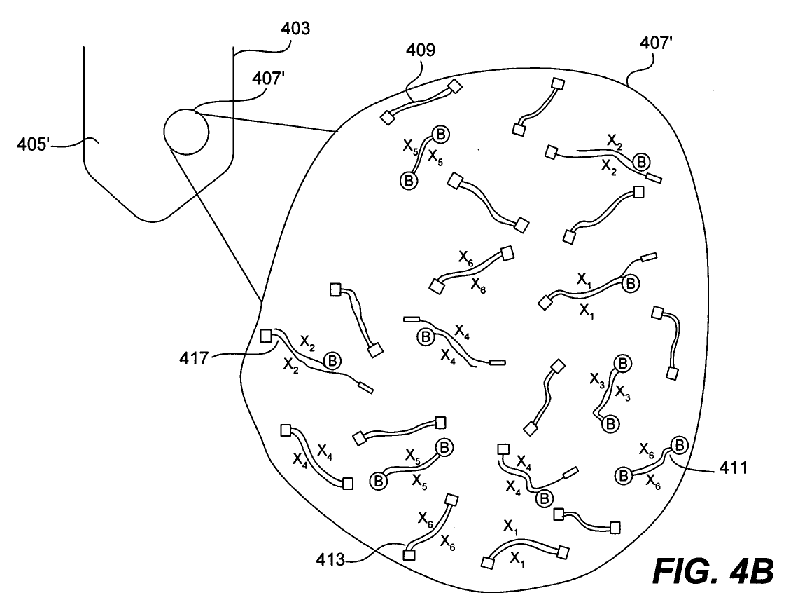

In an embodiment depicted in FIG. 5, the annealed mixture is contacted with beads having strepavidin moieties distributed over their surfaces. As shown, a plurality of beads 503 is added to the annealed mixture 405′. Initially, the individual beads have no immobilized selection probes. But they do have streptavidin moieties distributed over their surfaces as indicated by the “S”s on individual beads 505 shown in FIG. 5. After remaining in solution for a period of time, the beads capture some of the selection probes in solution. Some captured probes have annealed with target fragments as shown in FIG. 5; see bead 505′.

In a specific embodiment, the contact between the solution and beads takes place for a period of about 30 minutes to 1 hour at a temperature of about 20° C. to 37° C. This allows sufficient time for the biotin and strepavidin moieties to link with one another and effectively immobilize the double-stranded sequences of the selection probe and the complementary DNA fragments.

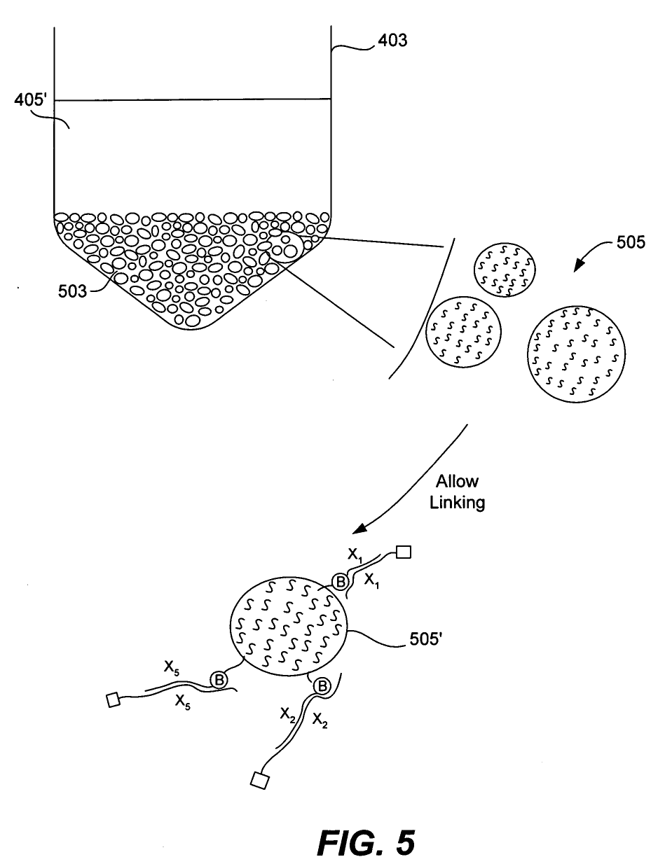

As indicated above, the sequence of the selection probe should be chosen to select target sequences including features of interest (e.g., one or more SNPs). Often the feature of interest will be centered in the probe sequence, but this is not necessary. In some cases, the feature of interest will be off-center or even outside the probe sequence. If the feature of interest is located outside the probe sequence, the probe sequence should be complementary to a region of the target sequence that is sufficiently proximate to the feature of interest that the probe will pick up fragments having such feature. These implementations are depicted in FIG. 6, which shows (a) a SNP or other feature of interest 603 centered in a selection probe 605, (b) the SNP 603 within a selection probe 607, but off center, and (c) the SNP 603 located outside the extent of a selection probe 609 but near one end of such probe.

At least a subset of the target fragments become attached to the solid substrate in the procedure outlined above. To enrich these fragments, the unattached fragments should be washed away or otherwise separated from the substrate. Recognizing that the target fragments are complementary to the immobilized probe sequences, various separation techniques will become apparent to those of skill in the art. For example, a two-stage washing procedure may be employed, with a first stage employed to remove DNA fragments that are on the substrate but are not bound through DNA-DNA interactions and a second stage performed under more stringent conditions to remove loosely hybridized sample nucleic acid strands, which may contain mismatches to one or more of the selection probes within a region that is otherwise complementary to the one or more selection probes.

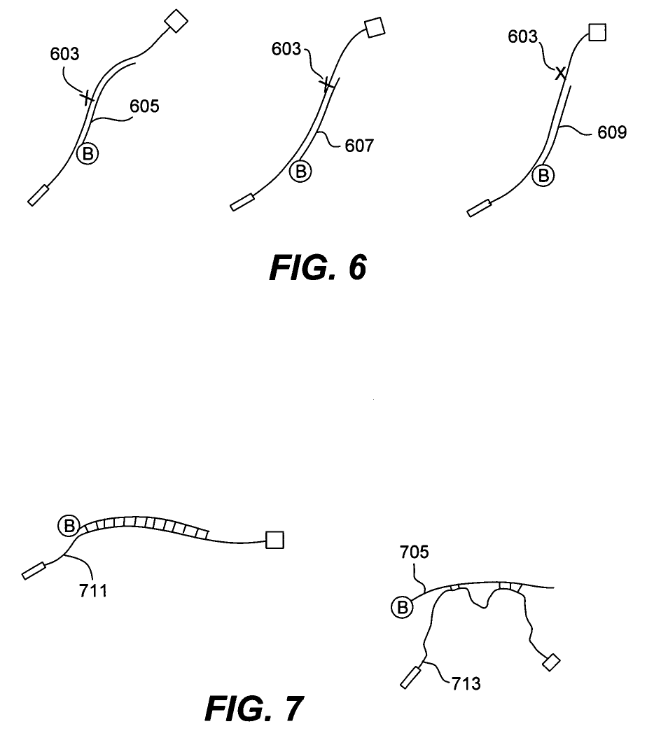

As an example, the first stage is conducted with 6×SSPE buffer at room temperature and the second stage is performed under most stringent conditions employing a lower salt concentration (representing more severe conditions) at a relatively higher temperature. For example, this may be employed with 0.2×SSPE at a temperature of about room temperature up to about 37° C. Again, this second wash will remove relatively loosely bound DNA fragments that may be partially complementary with the selection probes. FIG. 7 shows how fully complementary hybridized fragment 711 (which typically would not be removed by the second stage wash) and a partially hybridized fragment 713 (which much more likely would be removed by the second stage wash). Both fragments are shown hybridized to a selection probe 705.

After the non-annealed and loosely annealed sample fragments have been removed by the two washes described above, only the target DNA fragments should remain on the solid substrate. In other words, the substrate will at this point contain (ideally) only those nucleic acid fragments that are strongly complementary to the selection probes, which fragments are presumably target DNA fragments. Thus, the process to this point has effectively isolated the target fragments from the remainder of the sample. At this point, the target may be further processed or analyzed in a variety of ways as described below. Although the examples specifically describe analysis with DNA microarrays, it should be understood that the invention is not limited to this method.

As indicated in FIG. 1, block 113, the target DNA fragments are removed from the immobilized selection probes by, e.g., denaturation. In a specific example, this is accomplished by treatment with 0.15 M sodium hydroxide at room temperature. Thereafter, the solution is neutralized with 0.15 M hydrochloric acid. After denaturation, in which the target fragments have been removed from the substrate, the substrate itself (e.g., the beads) may be removed from the solution. The resulting solution contains the isolated and enriched target nucleic acid fragments.

Analysis of Isolated Target Fragments

In some embodiments, the isolated target fragments can be analyzed directly. For certain applications, however, they must first be further amplified and/or fragmented. As indicated above, the possibility of PCR suppression may limit the initial fragmentation procedure to production of fragments no smaller than approximately 300-400 base pairs. Such fragments may be too large to be effectively interrogated using a DNA microarray. Therefore, it may be necessary to further fragment the target stands.

Assuming that the enriched target fragments must be amplified (see operation 117 of FIG. 1), then PCR is performed using primers of the same sequence as were employed in the initial amplification (operation 107). The isolated target fragments will still have adaptor sequences attached, which can serve as the annealing site for PCR primers. In many cases, only a single primer sequence will be required for the second amplification because only a single adaptor sequence was employed earlier in the process (see operation 105 of FIG. 1). Typically, however, single-stranded primers will be employed here rather than the double-stranded adaptor sequences used in the initial amplification. The degree of amplification will depend upon the quantity of fragments that were captured and immobilized as well as the requirements of the sequence analysis technique. In a typical case, approximately 20 to 40 PCR cycles are employed.

After amplification, the isolated fragments are possibly too large to effectively hybridize with immobilized oligonucleotide probes on a DNA microarray. As indicated, it will then be desirable to further fragment the target strands. If a second fragmentation is employed, the conditions are chosen to produce fragments having a size that is appropriate for the analysis technique to be performed. For genotyping by a DNA microarray, the final fragment size is preferably between about 25 and 150 base pairs in length, or in some embodiments, between about 40 and 100. Contact with a DNase for an appropriate period of time may be employed to fragment the isolated target sequences and produce final fragments of this size. In other embodiments, the additional fragmentation is accomplished using shearing, restriction enzymes, etc as described above.

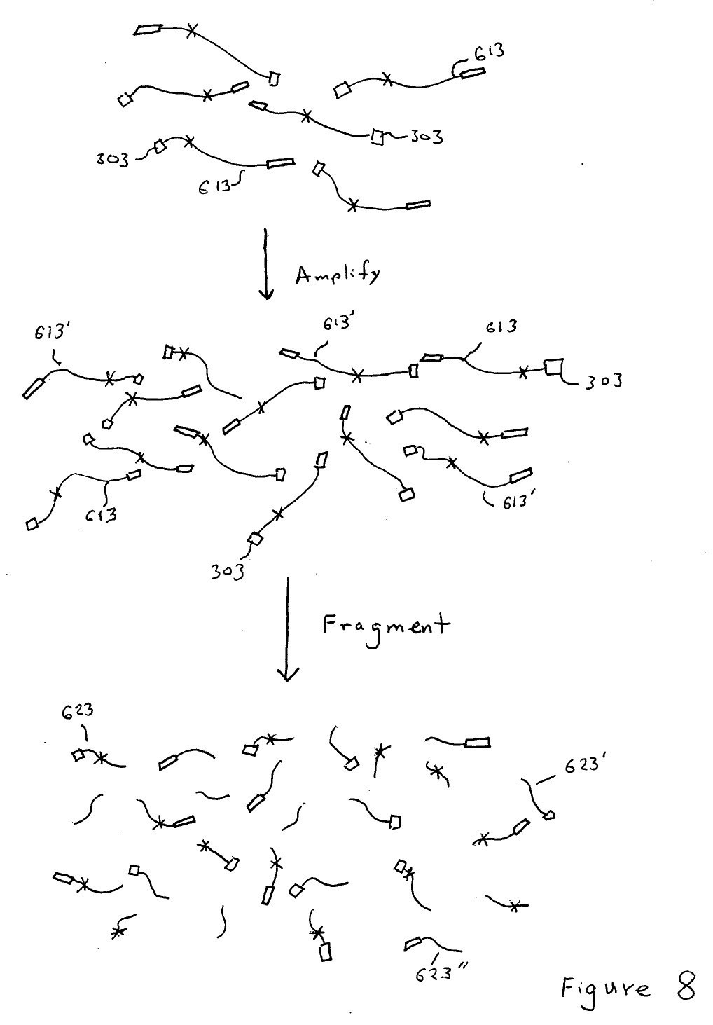

FIG. 8 follows the progression of the selected target fragments through a second round of amplification and fragmentation. As shown, target fragments 613 having adaptors 303 are amplified to produce additional copies 613′. The amplified target fragments are then fragmented to produce smaller target fragments 623, 623′, etc. As illustrated some of these fragments will not contain the target sequences of interest.

It is of course within the scope of the invention to use only a single fragmentation reaction. In such embodiments, the initial fragmentation produces fragments of an appropriate size for analysis of the isolated target fragments, e.g., genotyping using a conventional DNA microarray. Alternatively, the method employs a sequencing tool suitable for sequencing relatively large sequences (e.g., sequences of about 300 base pairs and larger). For example, a direct sequencing technique may be employed. Other embodiments employ sequencing platforms of Illumina, Inc. (San Diego, Calif.) and 454 Corporation (New Haven, Conn.). In general, the invention is not limited to any particular methodology or product for analyzing the target fragments isolated using this invention.

If a DNA microarray is employed to sequence the isolated target fragments, the fragments are first labelled and then contacted with the microarray under conditions that facilitate hybridization with the immobilized oligonucleotides. Any suitable label and labelling technique may be employed. Many widely used labels for this purpose provide fluorescent signals. In a specific example, terminal transferase enzyme is employed to label the fragments. After the labels are attached to the fragments and the fragments hybridize with the oligonucleotides on the microarray, the array may be stained and/or washed to further facilitate detection of the fragments bound to the array. The binding pattern on the array is then read out and interpreted to indicate the presence or absence of the various target sequences in the sample. In the case of SNP targets, a reader identifies the alleles present in the target sequences by virtue of, for example, (1) the known sequence and location of individual probes on the array; (2) knowing that a fragment is complementary to one or more probes on array; (3) therefore knowing the sequence of the fragment; and finally (4) therefore knowing the genotype of fragment. Labels, oligonucleotide microarrays, and associated readers, software, etc. are provided with various conventionally available DNA microarray products such as those commercially available from, e.g., Affymetrix, Inc., (Santa Clara, Calif.). As indicated, other methods are also suitable; for example, direct sequencing of the regions encoding each marker, creation of a library comprising the target sequences, use of the target sequences as probes in further experiments or methodologies, or use in functional assays in cell lines.

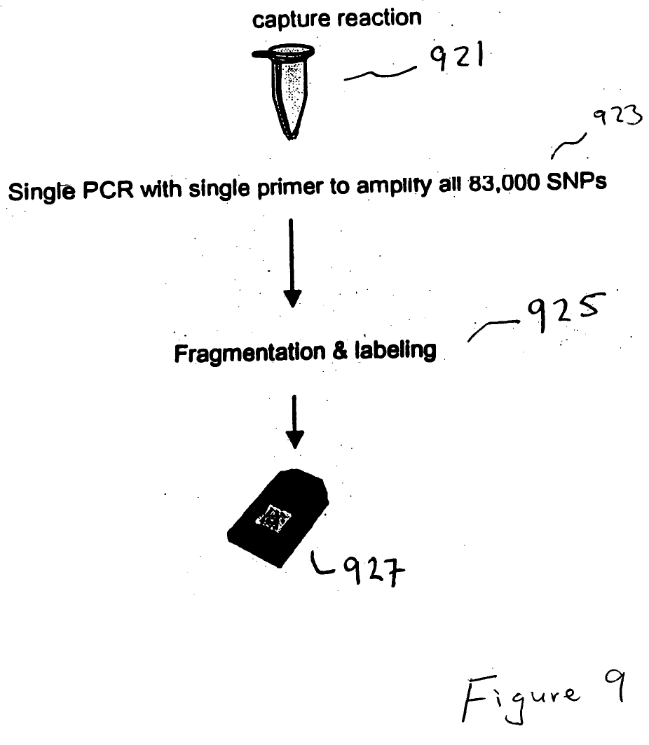

FIG. 9 shows a sequence of operations employed to sequence isolated target fragments in a specific embodiment as described above. In an operation 921, the free isolated target fragments are provided in a fluid medium. These were obtained by first washing the solid substrate to remove non-specific fragments and then releasing the specifically bound target fragments. 83,000 SNPs are represented in the target fragments. In an operation 923, the free target fragments are amplified using a single PCR with a single primer to amplify all 83,000 SNPs. Thereafter, in an operation 925, the fragments are further fragmented and labelled. Finally, in an operation 927, the labelled fragments are interrogated using a DNA microarray.

### EXAMPLE

Preparation of DNA Sample

Genomic DNA from human blood lymphocytes was isolated using commercially available kits following manufacturer-supplied protocols. Approximately 100 ng of genomic DNA was fragmented using DNase I in the presence of 1 mM MnCl₂. The fragmented DNA sizes range from about 200 bp to 1 kb when visualized by ethidium bromide staining after separation through agarose gel electrophoresis. The fragmented DNA was made blunt-ended by treatment with Pfu DNA polymerase at 65° C. in the presence of 200 mM dNTPs. Next, the blunt-ended fragments were ligated to a double-stranded adaptor at 4° C. using T4 DNA ligase for 16 hours. The ligated DNA was then used as template in a 20 to 24-cycle PCR reaction with the residual unligated adaptors from the ligation reaction serving as PCR primers. This reaction can be catalyzed by the Pfu DNA polymerase previously used to blunt the DNA fragment ends, or by other DNA polymerase enzymes added into the reaction. Typically, the PCR product ranges in size from about 300 bp to 1.2 kb, with the majority of the products at about 500-600 bp.

Annealing Reaction

Approximately 5 μg of the PCR product was mixed with 10 μg of COT-1 DNA and 100 μg of Herring Sperm DNA and the mixture was lyophilized to dryness by vacuum centrifugation. The dried DNA was then resuspended in a suitable hybridization buffer, such as 6×SSC or 6×SSPE, which may contain 50% formamide and/or hybridization accelerators such as 10% dextran sulfate or 10% polyethylene glycol. Approximately 50 ng of biotin-labeled DNA selection probe was added to the reaction and after denaturation at 95° C. for 2 min, the reaction was allowed to slowly cool to 37° C. over 2 hours. The annealing reaction was allowed to proceed at 37° C. for 20 to 36 hours.

Selection of Annealed DNA Fragments

100 μg of streptavidin coated 1 micron paramagnetic beads was added to the reaction and the biotinylated DNAs were allowed to bind to the beads at 37° C. for 30 min. Following binding, the beads were washed sequentially 2 times with 1 ml of 6×SSPE buffer at room temperature and 2 times with 1 ml of 0.2×SSPE at 37° C. for 30 min. The DNA captured on the beads was then released by incubation in 0.15M NaOH and the denatured DNA was neutralized by addition of an equal volume of 0.15M HCl. The neutralized DNA was then used in a PCR reaction with a single-stranded PCR primer having a DNA sequence corresponding to the ligated adaptor at the end of the DNA fragment. Amplified DNA was then purified, fragmented and end-labeled with Terminal transferase enzyme in preparation for microarray hybridization following standard procedures.

As illustrated by this example and the above description of a preferred embodiment, the invention provides a considerable reduction in complexity for processing large samples such as the human genome. As a point or reference, the human genome contains approximately 3 billion base pairs. Applying a set of 80,000 selection probes in accordance with this invention, can easily reduce the quantity of DNA to be analyzed by a factor of approximately 20; e.g., to about 80 million base pairs in the case of 500 bp sample fragments. Obviously, greater reductions in complexity will result when fewer selection probes are employed and/or when the sample fragments are smaller.

### Other Embodiments

The present invention has a broader range of implementation and applicability than described above. For example, while the methodology of this invention has been described in terms of genotyping using a DNA microarray, the inventive methodology is not so limited. For example, the invention could easily be extended to the selection and isolation of nucleic acids such as full-length cDNAs, mRNAs and genes, as well as other methods requiring complexity reduction such as gene expression analysis and cross-species comparative hybridizations. Those of ordinary skill in the art will recognize other variations, modifications, and alternatives.

It is to be understood that the above description is intended to be illustrative and not restrictive. It readily should be apparent to one skilled in the art that various embodiments and modifications may be made to the invention disclosed in this application without departing from the scope and spirit of the invention. The scope of the invention should, therefore, be determined not with reference to the above description, but should instead be determined with reference to the appended claims, along with the full scope of equivalents to which such claims are entitled. All publications mentioned herein are cited for the purpose of describing and disclosing reagents, methodologies and concepts that may be used in connection with the present invention. Nothing herein is to be construed as an admission that these references are prior art in relation to the inventions described herein. Throughout the disclosure various patents, patent applications and publications are referenced. Unless otherwise indicated, each is incorporated by reference in its entirety for all purposes.

What is claimed is:

1. A method of isolating target nucleic acid sequences from a nucleic acid sample, the method comprising:
(a) generating nucleic acid fragments from the sample;
(b) amplifying the nucleic acid fragments;
(c) exposing the amplified nucleic acid fragments to at least about 2,000 distinct selection probes in a single reaction medium under conditions promoting annealing between the selection probes and the amplified nucleic acid fragments that are complementary to the selection probes, wherein the selection probes have sequences complementary to the target nucleic acid sequences;
(d) removing the amplified nucleic acid fragments that are not strongly bound to the selection probes; and
(e) releasing annealed amplified nucleic acid fragments from the selection probes, wherein said annealed amplified nucleic acid fragments are said target nucleic acid sequences, thereby isolating said target nucleic acid sequences.

2. The method of claim 1, further comprising characterizing the nucleic acid sample on the basis of the target nucleic acid sequences released in (e).

3. The method of claim 2, wherein the characterizing is performed by applying the target nucleic acid sequences to a nucleic acid array.

4. The method of claim 3, further comprising:
amplifying the target nucleic acid sequences released in (e); and
labelling said target nucleic acid sequences prior to contacting them with said nucleic acid array.

5. The method of claim 4, further comprising further fragmenting the target nucleic acid fragments prior to labelling.

6. The method of claim 1, wherein fragmenting the nucleic acid sample produces nucleic acid fragments having an average size of between about 25 and about 2,000 base pairs.

7. The method of claim 6 wherein the average size of the nucleic acid fragments is about 500 base pairs.

8. The method of claim 1, wherein generating nucleic acid fragments in (a) produces nucleic acid fragments having an average size that allows genotyping on a nucleic acid array without further fragmentation.

9. The method of claim 1, wherein amplifying the nucleic acid fragments comprises performing a Polymerase Chain Reaction (PCR) on substantially all of the nucleic acid fragments produced in (a).

10. The method of claim 1, further comprising, prior to amplifying the nucleic acid fragments, attaching adaptors to the ends of the nucleic acid fragments, wherein the adaptors comprise sequences complementary to primers employed in the amplification operation.

11. The method of claim 10, wherein the adaptors each comprise the same sequence.

12. The method of claim 10, wherein the adaptors comprise dsDNA with ssDNA tail.

13. The method of claim 10, wherein excess adaptors that do not attach to the ends of the nucleic acid fragments serve as primers in amplifying the nucleic acid fragments.

14. The method of claim 10, wherein attaching the adaptors comprises ligating the adaptors to blunt ends of the nucleic acid fragments.

15. The method of claim 1, wherein the selection probes comprise moieties that facilitate linkage to a solid substrate.

16. The method of claim 15, further comprising linking the selection probes to a solid substrate, wherein at least a subset of the selection probes is annealed to the amplified nucleic acid fragments between operations (c) and (d).

17. The method of claim 16, wherein the solid substrate comprises a plurality of beads.

18. The method of claim 16, wherein removing the amplified nucleic acid fragments that are not strongly bound to the selection probes comprises washing the solid substrate to remove unbound nucleic acid fragments.

19. The method of claim 18, wherein washing the solid substrate comprises exposing the solid substrate to a solution under conditions that remove partially annealed amplified nucleic acid fragments from bound selection probes.

20. The method of claim 1, wherein exposing the amplified nucleic acid fragments to the distinct selection probes in a single reaction medium, comprises providing at least about 50,000 distinct selection probes, each complementary to a distinct target nucleic acid sequence, in the single reaction medium.

21. The method of claim 20, wherein the number of distinct selection probes employed in the single reaction medium is between about 50,000 about 10⁷.

22. The method of claim 1, wherein exposing the amplified nucleic acid fragments to distinct selection probes in a single reaction medium comprises exposing the amplified nucleic acid fragments to at least about 5,000 distinct selection probes in said single reaction medium.

23. The method of claim 22, wherein exposing the amplified nucleic acid fragments to distinct selection probes in a single reaction medium comprises exposing the amplified nucleic acid fragments to at least about 10,000 distinct selection probes in said single reaction medium.

24. A method of isolating target nucleic acid fragments from a mixture of target and non-target nucleic acid fragments, the method comprising:
(a) applying an adaptor sequence to the ends of the target and non-target nucleic acid fragments in the mixture, wherein the adaptor sequence comprises a sequence between about 15 and 40 base pairs in length, and is present in excess to the number of nucleic acid fragment ends;
(b) performing a polymerase chain reaction to amplify the target and non-target fragments, wherein no primer sequence is necessary to amplify the target and non-target fragments besides that provided by denaturing excess adaptors;
(c) contacting the amplified target and non-target fragments with a plurality of selection probes simultaneously, under conditions that promote annealing of the selection probes and the target nucleic acid fragments, wherein the selection probes comprise sequences complementary to sequences of the target nucleic acid fragments; and
(d) separating the non-annealed and partially-annealed non-target nucleic acid fragments from the annealed target nucleic acid fragments, which are bound to said selection probes, thereby isolating the target nucleic acid fragments.

25. The method of claim 24, wherein the adaptor sequence is a double-stranded nucleic acid sequence.

26. The method of claim 25, wherein the adaptor has a blunt end for attachment to the ends of the nucleic acid fragments.

27. The method of claim 26, wherein the adaptor has a sticky end having an overhang that is not complementary to itself, whereby the sticky ends of the adaptor do not anneal to one another.

28. The method of claim 26, wherein one strand of the adaptor is lacking a moiety necessary for ligation at the blunt end of the adaptor, whereby the blunt ends of the adaptor do not ligate to one another.

29. The method of claim 24, wherein the adaptor is present in an excess of between about 10-100 fold over the number of nucleic acid fragment ends.

30. A set of selection probes for use in simultaneously selecting target nucleic acid fragments from non-target nucleic acid fragments, wherein the set comprises:
at least about 10,000 distinct selection probes in a common medium, each selection probe having a sequence complementary to a distinct target sequence including a distinct SNP, all found in a single genome,
wherein each of the distinct selection probes is between about 20 and 1000 base pairs in length.

31. The set of selection probes of claim 30, wherein the individual selection probes of the set are double-stranded nucleic acid sequences.

32. The set of selection probes of claim 30, wherein the set comprises between about 10⁴ and 10⁸ distinct selection probes.

33. The set of selection probes of claim 30, wherein the set comprises between about 10⁴ and 10⁵ distinct selection probes.

34. The set of selection probes of claim 30, wherein each of the distinct selection probes further comprises a moiety, apart from the selection probe sequence, that facilitates binding to a solid substrate.

35. The set of selection probes of claim 34, wherein the moiety is biotin or streptavidin.

36. The set of selection probes as recited in claim 30, wherein the individual selection probes of the set are prepared by PCR reactions specific for the individual selection probes.

37. A kit for isolating target nucleic acid fragments from non-target nucleic acid fragments, the kit comprising:
the set of selection probes as recited in claim 34; and
a solid substrate comprising a surface feature for binding with the moiety on the selection probes and thereby facilitating immobilization of the selection probes on the substrate.

38. The kit of claim 37, further comprising primers and polymerase for amplifying the nucleic acid fragments.

39. The kit of claim 37, further comprising a nucleic acid array comprising sequences complementary to the target nucleic acid fragments.

40. The kit of claim 37, wherein the solid substrate comprises beads.
